(19) United States
(12) Patent Application Publication
(10) Pub. No.: US 2003/0055320 A1
(43) Pub. Date: Mar. 20, 2003

(54) Tissue retractor

(76) Inventors: G. Grady McBride, Winter Park, FL (US); William A. Cottle, Vancouver (CA)

Correspondence Address: ERIC B. MEYERTONS, CONLEY, ROSE & TAYON, P.C., P.O. BOX 398, AUSTIN, TX, 78767-0398, US

(21) Appl. No.: 09/954,971

(22) Filed: Sep. 18, 2001

(51) Int. Cl. A61B 1/32

(52) U.S. Cl. 600/217

(57) ABSTRACT

A retraction apparatus and method for separating human tissue during surgical procedures is described for use. The retraction apparatus may include two elongated members, a first end having two protrusions located on the first end and on the mid-shaft, a second end having grips, a retainer, and a fastener. The retraction apparatus includes protrusions for coupling to human tissue. The retraction apparatus includes grips that enable separation of the first ends of the elongated members when the second ends are brought together. The grips may be attached to the retainer, located at the second end, for fixing the position of the first ends. Locking the retraction apparatus into a fixed position consists of engaging a fastener attached to the grips to the retainer such that the first ends substantially resist any force to move or disengage from human tissue.

### BACKGROUND OF THE INVENTION

1. Field of the Invention

The present invention generally relates to medical instruments. An embodiment of the invention relates to a surgical tissue retractor that is capable of widening a surgical incision, holding the incision open without rotation of the retractor during a surgical procedure, and inhibiting accidental release of the retractor during the surgical procedure.

2. Description of Related Art

A retractor may be used to widen a surgical incision or opening. The retractor may move and separate tissue to provide access to areas underlying the tissue. The tissue may include, but is not limited to skin, fat, and/or muscle. There are many different types of retractors.

One type of retractor includes two elongated members having grips, a hinged midsection connecting the two elongating members, and singular protrusions near ends of the elongated members. A holding mechanism adjacent to the hinged mid-section allows the retractor to maintain tissue in a spread apart position when the grips are released. The single protrusion on the members may allow the retractor to rotate upwards from a desired initial position during use. Forcing the retractor back to the initial position during a medical procedure may be distracting and annoying to a surgeon or surgical team. The grips of retractors may be small. The small grips may make applying enough force to the retractor to separate tissue difficult. The location of the holding mechanism may be inconvenient during use. The holding mechanism may also require a significant amount of force to lock and unlock the retractor such that it may become tiresome and difficult to use during a surgical procedure.

Another type of retractor includes two elongated members having grips, a hinged mid-section connecting the two members, and multiple protrusions near ends of the elongated members. A holding mechanism adjacent to the hinged mid-section allows the retractor to maintain tissue in a spread apart position when the grips are released. The multiple protrusions near the ends of the elongated members are of equal size to provide placement of structures such as tissue and/or vessels therein. The size of the grips and the location of the holding mechanism impose the same difficulties described above.

### SUMMARY OF THE INVENTION

A retractor may be used to separate tissue adjacent to an incision or opening during a medical procedure. The retractor may include elongated members having multiple protrusions on an end of each member, handgrips on another end of each member, a hinge section pivotally connecting the members, spring members, and a retainer.

The elongated members may include a first end and a second end opposite the first end. The multiple protrusions may be located proximate the first ends of each elongated member and may extend from each member to provide a means for engaging tissue structure. The placement of the protrusions will be located as to prevent the rotational movement of the retractor from a desired position once engaged to tissue structure. The hinge section may be located between the first end and the second end such that the elongated members may rotate about a pivot point of the hinge section. The spring members may be affixed to the retractor to provide a force to the second ends of the elongated members. The handgrips may be located proximate the second ends of the elongated members and may allow a substantially high separating force to tissue structure. The grips may be of a size to allow the use of one or multiple hands for grasping and/or applying force. The retainer serves to fix the retractor in a desired spread apart position after the handgrips are released. The retainer may be form-locking or force-locking, and is designed and located to provide ease of engagement and disengagement.

An embodiment of the retractor may include elongated members, which may extend from a first end to a second end located opposite the first end. A hinge section may be located between the first ends and the second ends of each elongated member, which connects the separate members and provides a pivot point of the members. The elongated members may be shaped and the hinge section may be located such that the first ends of the elongated members spread apart from each other from the hinge section when the second ends of the elongated members are forced together. Alternately, the first ends of the elongated members come together from the hinge section when the force on the second ends of the elongated members is released and the second end move apart from each other from the hinge section. The first ends of the elongated members may be inserted into an incision or opening produced during a surgical operation to move or separate tissue.

The grips may be located proximate the second end of each elongated member. The grips may be sized such that a single hand or multiple hands can be used for grasping and applying force to the members of the retractor. In addition, spring members may be located proximate the second end. The spring members may be as a cantilever, coil springs, or leaf springs, and may provide a force to maintain the separation of the second ends of the elongated members when the retractor is not engaged to tissue structure. A force may be applied to the grips of the elongated members to force the second ends of the elongated members together. As the force is transferred through the pivot of the hinge section, the first ends of the elongated members spread apart from one another and may contact adjacent tissue of a surgical incision or opening.

At least two protrusions may be located proximate the first end of each elongated member. The protrusions may extend from the elongated members to engage tissue and provide a means for anchoring or locking the first end of each elongated member to a tissue structure. The protrusions may be located proximate each other along the length of the first end of each elongated member. Each protrusion on a single elongated member may engage tissue to serve as a separate anchor such that rotational movement of the retractor from a desired position is inhibited or substantially reduced.

Applying additional force on the grips to bring the second ends of the elongated members closer together results in further separation of the first ends of the elongated members and tissue structure in which the first ends may be in contact. Once a desired separation of the tissue structure is achieved, the retainer may be engaged to fix the separation of the first ends of the elongated members and tissue structure. The retainer may be, but is not limited to, a ratchet mechanism having serrations or teeth which allow ease in forcing the second ends of the elongated members together, but resists the force of the spring members to inhibit separation of the second ends of the elongated members until the user disengages the ratchet. The retainer may be located at the base of the grips and may be rotatably coupled to the second end of an elongated member. The location of the retainer may also allow convenient access to the retainer by a member of a surgical team who may not be grasping the retractor.

The retainer may include a fastener (i.e., latch or clasp) coupled to the second end of another elongated member to lock the retainer in a desired spread apart position to further inhibit unwanted disengagement during a medical procedure.

An embodiment of a method using the surgical tissue retractor may include inserting the retractor into a surgical incision or opening. A force may be applied to the second ends of the elongated members to separate the first ends of the elongated members, which contacts and separates tissue adjacent to the incision or opening. The retainer may be locked in place by the fastener at the desired spread apart position during the medical procedure.

### BRIEF DESCRIPTION OF THE DRAWINGS

Other objects and advantages of the invention will become apparent upon reading the following detailed description and upon reference to the accompanying drawings in which:

FIG. 1 depicts a side view of an embodiment of a retractor;

FIG. 2 depicts a top view of an embodiment of a retractor;

FIG. 3 depicts a front view of a first end of a retractor comprising of two protrusions;

FIG. 4 depicts a side view of a fastener for a retractor;

FIG. 5 depicts a top view of a fastener for a retractor; and

FIG. 6 depicts a bottom view of a fastener for a retractor.

While the invention is susceptible to various modifications and alternative forms, specific embodiments thereof are shown by way of example in the drawing and will herein be described in detail. The drawings may not be to scale. It should be understood, however, that the drawings and detailed description thereto are not intended to limit the invention to the particular form disclosed, but on the contrary, the intention is to cover all modifications, equivalents, and alternatives falling within the spirit and scope of the present invention as defined by the appended claims.

### DETAILED DESCRIPTION OF PREFERRED EMBODIMENTS

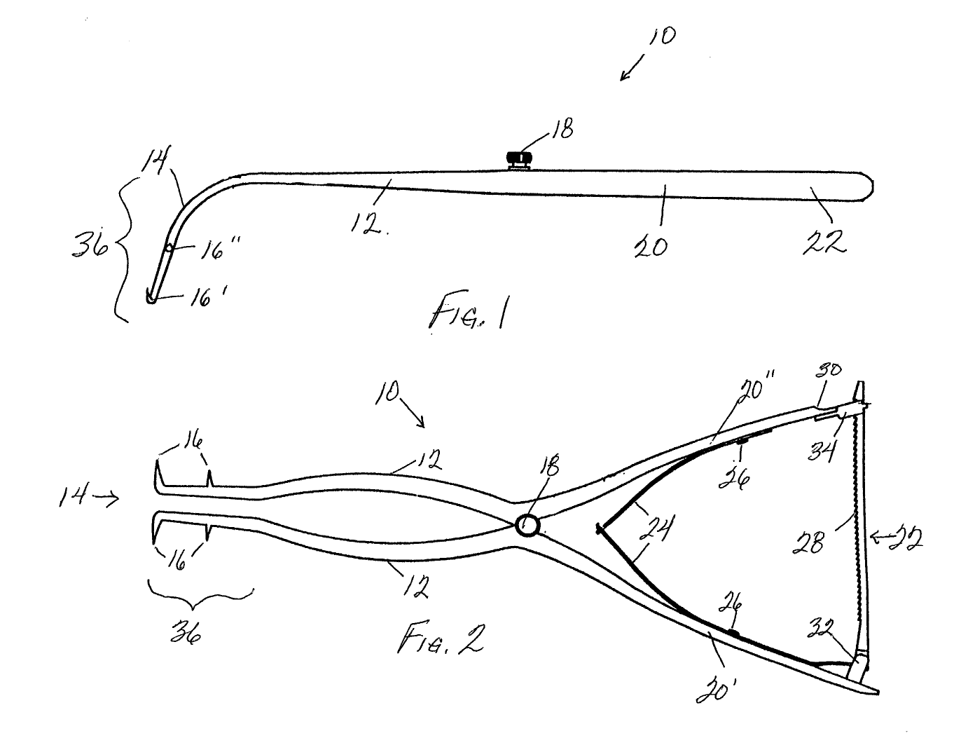

A side and top view of an embodiment of a retractor 10 is depicted in FIGS. 1 and 2, respectively. Retractor 10 may include two elongated members 12, each having a first end 14. Protrusions 16 may be located proximate the first end 14 of the elongated members 12. A hinge section 18 may connect the two elongated members 12 and provide a pivot for rotation of the members. Grips 20 may be located proximate a second end 22 opposite the first end 14 of each elongated member 12. A spring member 24 may be affixed at a point 26 to the second ends 22 of the elongated members 12. The spring members 24 may apply forces to the grips 20 to maintain separation of the second ends 22 during use. A retainer 28 may include serrations, as shown in FIG. 2, and a pawl 30 may be located at the second end 22 of an elongated members 12 to engage the serrations of the retainer 28. The retainer 28 may pivot at point 32 to allow ease of engagement and disengagement of the retainer 28. The retainer may include a locking mechanism or fastener 34 to further inhibit unwanted disengagement of the retainer 28 from the pawl 30.

FIG. 1 depicts a side view of the retractor 10. The retractor 10 may include two elongated members 12 with grips 20, a first end 14, and a second end 22, and a pivot point 18. The elongated members 12 may include a mid-shaft 36 proximate the first end 14 which curves downward. Positioning of the retractor 10 during use may be in a substantially horizontal position parallel to the area to be opened and cleared, such that the mid-shaft 36 of the elongated members 12 points downward, or substantially perpendicular, to the retractor.

The mid-shaft 36 may be inserted into an incision or opening. With one or both hands, a user may bring each of the second ends 22 of the elongated members 12 together by applying a force to grips 20. As the second ends 22 of the elongated members 12 converge, mid-shaft 36 may begin to separate adjacent tissue structure. As depicted in FIG. 3, protrusions 16 may be located on each mid-shaft 36. The protrusions 16 may engage tissue structure adjacent to mid-shaft 36, anchoring the retractor 10 to the tissue structure. The protrusions 16 may have sharpened points or blunt ends. A first protrusion 16′ may be proximate the first end 14 and may be at least the same length or greater in length than a second protrusion 16″. The second protrusion 16″ may be located at a distance further from the first end 14 than the first protrusion 16′ along the mid-shaft 36. The protrusions 16 may be positioned relative to each other on mid-shaft 36 such that the protrusions 16 inhibit rotation about the pivot created by the other protrusions 16 and the engaged tissue structure. Once protrusions 16 are engaged to tissue structure, applying additional force to grips 20 to converge second ends 22 of elongated members 12 may further separate first ends 14 and adjacent tissue structure to access areas of surgical interest.

A retainer 28 may be rotationally coupled at a pivot point 32 to the grip 20′ proximate the second end 22 of an elongated member 12 of the retainer 28. The retainer 28 may be rotated towards or away from the grip 20″ proximate the second end 22 of the other elongated member 12. The retainer 28 may contact a pawl 30 proximate the grip 20″. The pawl may be an end of grip 20″ of an elongated member 12. The pawl 30 may interact with the retainer 28 to inhibit separation of the grips 20′ and 20″ from each other if a user releases the grips. The retainer 28 and pawl 30 may be positioned at other locations along lengths of the grips 20. The retainer 28 may include a number of serrations as shown in FIG. 2. The pawl 30 may engage the serrations. Interaction of the serrations with the pawl 30 may allow the grips 20′ and 20″ to move towards each other if a user grasps the grips and forces the grips towards each other. The interaction of the pawl 30 and the serrations of the retainer 28 may counter the effect of spring members 24 that are coupled to the grips 20′ and 20″.

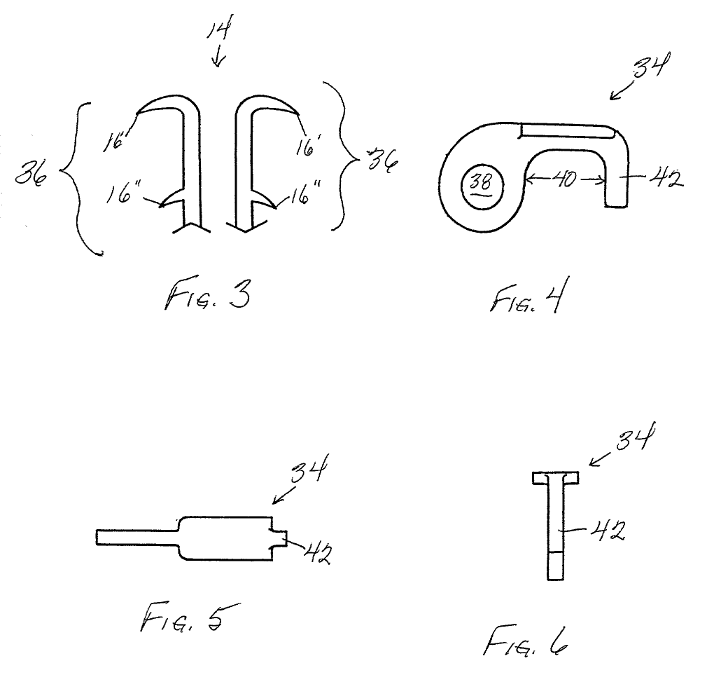

The retainer 28 may further include a locking mechanism or fastener 34 proximate pawl 30. A fastener 34 may be used to inhibit undesired rotation of the retainer 28 away from the grip 20″ during use. As depicted in FIGS. 4-6, the fastener 34 may include a pivot 38, a gap 40, and a lip 42. The fastener 34 may be rotated about pivot 38 to allow the retainer 28 to reside in gap 40. The lip 42 of the fastener 34 may inhibit movement of the retainer 28 away from pawl 30, preventing unwanted disengagement of the retainer 28 during use caused by accidental jostling of the retractor 10 when a user is not holding the grips 20.

A method for separating human tissue may include forming an incision or opening in a patient for inserting a retractor 10. The retractor 10 may include two elongated members 12 pivotally connected together at a point 18. Each elongated member may have a first end 14 and a second end 22. The first end 14 of each elongated member 12 may be inserted into the opening and includes at least two protrusions 16 to engage adjacent tissue. The second ends 22 of the elongated members 12 include grips 20 for forcing the second ends 22 together from the pivot point 18. Convergence of the second ends 22 causes the first ends 14 of the elongated members 12 to engage and separate the adjacent tissue. The protrusions 16 substantially inhibit rotation of the retractor 10 during use. A retainer 28 may be used to maintain the distance of the second ends of the elongated members 12 when the grips are released. A fastener 34 may be used to lock the retainer 28 at a desired spaced apart position. To remove the retractor 10, the fastener 34 may be rotated away from the retainer 28 and the retainer rotated at pivot point 32 proximate grip 20′ away from a pawl 30 proximate the grip 20″. Spring members 24 proximate the second ends 22 of the elongated members 12 provides a force to cause the second ends of the elongated members to diverge to an initial position. Divergence of the seconds ends causes the first ends 14 of the elongated member 12 to come together so that the retractor 10 may be removed from the incision or opening.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A retraction apparatus comprising:
a first member and a second member pivotally coupled, wherein each member comprises a first end and a second end; and
two protrusions located proximate the first end of each member, the protrusions configured to couple to tissue during use;
wherein movement of the second ends of the members forces the first ends to engage and separate the tissue during use.

2. The apparatus of claim 1, wherein the tissue comprises muscle.

3. The apparatus of claim 1, further comprising handgrips proximate the second ends of the members, the handgrips configured for a human hand to grasp.

4. The apparatus of claim 1, further comprising spring members coupled to the members.

5. The apparatus of claim 4, wherein the spring members apply a separating force to the second ends of the members.

6. The apparatus of claim 1, further comprising a retainer proximate the second ends of the members, the retainer configured to maintain a spread apart position between the first ends of the members during use.

7. The apparatus of claim 6, the retainer further comprising serrations, the serrations configured to engage a pawl proximate the second end of one of the members.

8. The apparatus of claim 6, wherein the retainer is pivotally coupled to the second end of the first member.

9. The apparatus of claim 6, further comprising a fastener, the fastener configured to inhibit movement of the first ends from a set separation distance.

10. The apparatus of claim 9, wherein the fastener is pivotally coupled to the second member.

11. The apparatus of claim 1, wherein one protrusion is located on a tip of the first ends of each member.

12. The apparatus of claim 11, wherein the other protrusions are located further from the tips along the length of the first ends of the members.

13. The apparatus of claim 11, wherein the protrusion located on the tips of the first ends of each member is at least the same length as the other protrusion proximate the first ends of each member.

14. The apparatus of claim 11, wherein the protrusion located on the tips of the first ends of each member is greater in length than the other protrusion proximate the first ends of each member.

15. The apparatus of claim 1, wherein at least one protrusion comprises a point.

16. The apparatus of claim 1, wherein the protrusions comprise blunt ends.

17. The apparatus of claim 1, wherein the protrusions comprise points.

18. A retraction apparatus comprising:
a first member and a second member pivotally coupled, wherein each member comprises a first end and a second end; and
a grip located between a pivot point and the second ends of the members, the grip configured for a human hand to grasp and move the second end of one member towards the second end of the second member;
wherein movement of the second ends of the members forces the first ends to engage and separate the tissue during use.

19. The apparatus of claim 18, wherein the tissue comprises muscle.

20. The apparatus of claim 18, further comprising spring members coupled to the members.

21. The apparatus of claim 20, wherein the spring members apply a separating force to the grips.

22. The apparatus of claim 18, further comprising a retainer proximate the second ends of the members, the retainer configured to maintain a spread apart position between the grips during use.

23. The apparatus of claim 22, the retainer further comprising serrations, the serrations configured to engage a pawl proximate the second end of one of the members.

24. The apparatus of claim 22, wherein the retainer is pivotally coupled to the second end of the first member.

25. The apparatus of claim 22, further comprising a fastener, the fastener configured to inhibit movement of the first ends from a set separation distance.

26. The apparatus of claim 25, wherein the fastener is pivotally coupled to the second member.

27. The apparatus of claim 18, wherein the first ends of each member comprise two protrusions.

28. The apparatus of claim 27, wherein the protrusions are configured to couple to the tissue during use.

29. The apparatus of claim 27, wherein one protrusion is located on a tip of the first ends of each member.

30. The apparatus of claim 29, wherein the other protrusions are located further from the tips along the length of the first ends of the members.

31. The apparatus of claim 29, wherein the protrusion located on the tips of the first ends of each member is at least the same length as the other protrusion proximate the first ends of each member.

32. The apparatus of claim 29, wherein the protrusion located on the tips of the first ends of each member is greater in length than the other protrusion proximate the first ends of each member.

33. The apparatus of claim 27, wherein at least one protrusion comprises a point.

34. The apparatus of claim 27, wherein the protrusions comprise blunt ends.

35. The apparatus of claim 27, wherein the protrusions comprise points.

36. A retraction apparatus comprising:
a first member and a second member pivotally coupled, wherein each member comprises a first end and a second end;
a grip located between a pivot point and the second ends of the members, the grip configured for a human hand to grasp and move the second end of one member towards the second end of the second member;
a retainer pivotally coupled to the second end of the first member, the retainer configured to maintain a spread apart position between the first ends of the members during use; and
a fastener pivotally coupled to the second end of the second member, the fastener configured to lock the retainer in a desired spread apart position during use;
wherein movement of the second ends of the members forces the first ends to engage and separate the tissue during use.

37. The apparatus of claim 36, wherein the tissue comprises muscle.

38. The apparatus of claim 36, further comprising spring members coupled to the members.

39. The apparatus of claim 38, wherein the spring members apply a separating force to the grips.

40. The apparatus of claim 36, the retainer comprising serrations, the serrations configured to engage a pawl proximate the second end of one of the members.

41. The apparatus of claim 36, wherein the first ends of each member comprise two protrusions.

42. The apparatus of claim 41, wherein the protrusions are configured to couple to the tissue during use.

43. The apparatus of claim 41, wherein one protrusion is located on a tip of the first ends of each member.

44. The apparatus of claim 43, wherein the other protrusions are located further from the tips along the length of the first ends of the members.

45. The apparatus of claim 43, wherein the protrusion located on the tips of the first ends of each member is at least the same length as the other protrusion proximate the first ends of each member.

46. The apparatus of claim 43, wherein the protrusion located on the tips of the first ends of each member is greater in length than the other protrusion proximate the first ends of each member.

47. The apparatus of claim 41, wherein at least one protrusion comprises a point.

48. The apparatus of claim 41, wherein the protrusions comprise blunt ends.

49. The apparatus of claim 41, wherein the protrusions comprise points.

50. A method of retraction comprising:
forming an incision in tissue;
by inserting a retraction apparatus in the incision, the apparatus comprising:
a first member and a second member pivotally coupled, wherein each member comprises a first end and a second end;
two protrusions located proximate the first end of each member, the protrusions configured to couple to the tissue during use;
a grip located between a pivot point and the second ends of the members, the grip configured for a human hand to grasp and move the second end of one member towards the second end of the second member;
a retainer pivotally coupled to the second end of the first member, the retainer configured to maintain a spread apart position between the first ends of the members during use; and
a fastener pivotally coupled to the second end of the second member, the fastener configured to lock the retainer in a desired spread apart position during use;
moving the second ends of the members together to force the first ends to engage and separate the tissue; and
locking the apparatus in the desired position.

51. The method of claim 50, wherein the tissue comprises muscle.

52. The method of claim 50, wherein the apparatus further comprises spring members coupled to the elongate members.

53. The method of claim 52, wherein the spring members apply a separating force to the grips.

54. The method of claim 50, the retainer comprising serrations, the serrations configured to engage a pawl proximate the second end of the second member.

55. The method of claim 50, wherein one protrusion is located on a tip of the first ends of each member.

56. The method of claim 55, wherein the other protrusions are located further from the tips along the length of the first ends of the members.

57. The method of claim 55, wherein the protrusion located on the tips of the first ends of each member is at least the same length as the other protrusion proximate the first ends of each member.

58. The method of claim 55, wherein the protrusion located on the tips of the first ends of each member is greater in length than the other protrusion proximate the first ends of each member.

59. The method of claim 50, wherein at least one protrusion comprises a point.

60. The method of claim 50, wherein the protrusions comprise blunt ends.

61. The method of claim 50, wherein the protrusions comprise points.

62. A retraction apparatus comprising:
a first member and a second member;
at least two protrusions located proximate a first end of the members;
wherein moving second ends of the members together moves the first ends to separate tissue.

63. A method of retraction comprising;
forming an incision in tissue;
inserting first ends of a retraction apparatus into the incision;
moving second ends of the apparatus together to move the first ends to separate the tissue.
